(19) United States
(12) Patent Application Publication
(10) Pub. No.: US 2017/0306283 A1
(43) Pub. Date: Oct. 26, 2017

(54) TRYPSIN-FREE CELL STAMP SYSTEM AND USE THEREOF

(71) Applicant: COLLEGE OF MEDICINE POCHON CHA UNIVERSITY INDUSTRY -ACADEMIC COOPERATION FOUNDATION, Pocheon-si, Gyeonggi-do (KR)

(72) Inventors: Han Soo PARK, Seongnam-si (KR); Ah Yeon RYANG, Incheon (KR); Ha Ram LEE, Seoul (KR); Soo Hong LEE, Seongnam-si (KR); In Bo HAN, Seongnam-si (KR); Byoung Ju KIM, Suwon-si (KR)

(73) Assignee: COLLEGE OF MEDICINE POCHON CHA UNIVERSITY INDUSTRY -ACADEMIC COOPERATION FOUNDATION, Pocheon-si, Gyeonggi-do (KR)

(21) Appl. No.: 15/520,673

(22) PCT Filed: Oct. 20, 2015
(86) PCT No.: PCT/KR2015/011045

§ 371 Date: Jun. 5, 2017

(30) Foreign Application Priority Data
Oct. 20, 2014 (KR) 10-2014-0141828

(51) Int. Cl. C12M 1/12; C12N 5/00; C12N 5/0735

(52) U.S. Cl. CPC C12M 25/14; C12N 5/0606; C12N 5/0062; C12N 5/0068; C12N 5/0025; C12N 2531/00; C12N 2533/50; C12N 2533/54

(57) ABSTRACT

The present invention relates to a trypsin-free cell stamp system and a use thereof. According to the present invention, an increase in the passage number of stem cells can be prevented compared with conventional methods of isolating cells from a cell culture dish, while providing a support, which is an essential condition of cell growth, by introducing the trypsin-free cell stamp system, and cells can be continuously supplied for a polymer-based fiber support without an additional subculture process since the empty space of a cell culture dish is filled as times passes. In addition, the artificial effects on cells can be minimized since the cells migrate to a polymer-based nano/micro-fiber support without other external stimulation, and thus the potency of stem cells is increased, thereby inducing more effective differentiation, such that the present invention, as a cell therapeutic agent, can be utilized in general fields of regenerative medicine and tissue engineering.

### TECHNICAL FIELD

The present invention relates to a trypsin-free cell stamp system, and a method of supporting cells, a method of culturing cells, and a method of transplanting cells by using the same. More particularly, the present invention relates to a method of culturing cells and a method of transplanting cells, in which anchorage-dependent cells are cultured on a support, and thus an additional subculture process and trypsin treatment are not required, unlike a known method of culturing cells.

### BACKGROUND ART

The development of regenerative medicine and tissue engineering has become an ideal way of overcoming limitations due to lack of tissues or organs as an alternative. According to tissue engineering techniques, specific cells which are isolated from a patient and cultured are adhered to a support made of a biocompatible/biodegradable material, and then developed into tissues by biochemical stimulation using biologically active factors or physical stimulation using a bioreactor. In other words, artificially engineered organs are similar to our body tissues, and thus have a greater potential as a substitute for autologous transplantation.

According to traditional tissue engineering methods, cells are isolated from a specific tissue and mass-cultured to obtain a sufficient number of cells, which are evenly cultured on a porous support. This support on which cells are cultured is then transplanted into the body, and replaces functions of damaged tissues or organs. Until now, artificial blood vessels, artificial urethras, artificial bladders, artificial cartilage, etc. have been manufactured by tissue engineering methods, and successfully applied in clinical practice. Many other tissues and organs are being actively studied. A successful series of studies are continuously being conducted on bio-hybrid tissues and organs which are obtained by hybridization of bio-organs, for example, hybridization of bone and joint, in addition to single bio-organs such as skin, bone, cartilage, peripheral and central nerves, tendons, muscle, a cornea, a bladder, a urethra, and a liver, which are obtained by using polymers, ceramics, metals, and complex materials, and hybridization thereof.

Cell therapy is a technique of culturing and manipulating cells extracted from a patient under a specific environment and then injecting the cells back into the patient, or of preparing human tissue by using cells and then using the tissue for therapy. A representative field of cell therapy is the treatment of cancer by using immune cells such as white blood cells, or regeneration of cartilages or cardiac muscles by using stem cells.

Traditional tissue engineering techniques require a series of processes of separating cells from a patient's tissue, culturing the cells, and propagating the cells. However, in many cases, it is not easy to obtain a source of cells, and there are limitations in proliferating isolated cells or maintaining a phenotype of cells. Practically, it takes much time and effort to standardize a series of processes which are involved in cell culture.

Meanwhile, the use of stem cells has been suggested as a way of effectively addressing these problems. However, a method of using stem cells is still required for a series of processes such as cell isolation, culturing, and proliferation, and in order to apply the method to clinical fields, a technique for differentiating stem cells into desired cells is also required. Stem cells, once transplanted, are believed to differentiate into cells similar to those of a transplantation site, according to the surrounding environment, but damaged tissues are often composed of fibrous tissue rather than normal tissue. Therefore, differentiation of transplanted stem cells into desired cells is an important factor for clinical success. In order to control differentiation of stem cells, a method of creating a transplantation site environment in which the stem cells differentiate, or a method of predetermining the fate of stem cells prior to transplantation and then transplanting the stem cells, may be attempted. When autologous chondrocytes are used, an amount of cells to be collected is very limited, and there is a limitation in which a change of cell phenotype occurs due to cell dedifferentiation during ex-vivo culture.

In culturing such cells, a subculture method is generally used. However, as cells continue to divide, they stop growing because there is not enough space to grow on a culture dish. To keep the cells proliferating, a cumbersome process of detaching a portion of cells from the culture dish and transferring the cells onto a new culture dish is required, and there are concerns regarding reduction in productivity and quality of the cultured cells due to an increase of the passage number of the cells. In addition, a large amount of media and a large number of culture dishes are required to obtain a large number of cells, and trypsin treatment destroys extracellular matrices, thus causing damage to cells. For these reasons, in the prior art, a temperature-sensitive microcarrier was prepared for more efficient cell culture in order to simplify the cell culture method and to minimize cell damage. However, on the whole, the prior art was still limited by subculturing (Yang, Hee-Seok, Tissue Engineering and Regenerative Medicine, 6(13), 1262-1267, 2009).

Accordingly, the present inventors found that affinity between a polymer-based nano/microfiber support and cells is used to induce spontaneous migration of cells to the polymer-based fiber support by a stamping method without physical and chemical treatment, so that an increase in the passage number of cells may be prevented, and repeated cell culture is possible without additional subculturing from one culture dish, thereby completing the present invention.

### DETAILED DESCRIPTION OF THE INVENTION

### Technical Problem

An object of the present invention is to provide a new concept of a trypsin-free cell stamp system, and a method of culturing cells and a method of transplanting cells by using the trypsin-free cell stamp system, wherein the trypsin-free cell stamp system includes a nano/microfiber support prepared by using a natural polymer and a synthetic polymer, enables migration of cells to the fiber support without cell damage due to external stimulation, and has a positive effect on differentiation potential of stem cells without an increase in the passage number, and therefore, is easy, simple, and efficient, as compared to a known method of inoculating cells on a support.

### Technical Solution

To achieve the above objects, an aspect of the present invention provides a trypsin-free cell stamp system. In the system, the stamp may include a polymer-based nano/microfiber support.

Another aspect of the present invention provides a method of supporting cells by using the trypsin-free cell stamp system, the method including the steps of: (a) seeding cells in a cell culture plate, and then culturing the cells; and (b) supporting the cultured cells by contacting the cultured cells with a stamp.

Still another aspect of the present invention provides a method of culturing cells by using the trypsin-free cell stamp system, the method including the steps of: (a) seeding cells in a cell culture plate, and then culturing the cells; (b) supporting a portion of the cultured cells on a stamp by contacting the cultured cells with the stamp; and (c) culturing non-supported cells which remain on the cell culture plate.

Still another aspect of the present invention provides a method of transplanting cells by using the trypsin-free cell stamp system, the method including the steps of: (a) seeding cells in a cell culture plate, and then culturing the cells; (b) supporting the cultured cells on a stamp by contacting the cultured cells with the stamp; (c) separating the support, on which the cells are supported, from the stamp; and (d) transplanting the separated support into a living body.

### Advantageous Effects of the Invention

The present invention may provide a relatively simple method of culturing cells, in which an additional subculture process is not required since anchorage-dependent cells are transferred as they are onto a support, and cultured thereon, unlike known methods of culturing cells. Introduction of the trypsin-free cell stamp system according to the present invention may provide the support essential for cell growth while preventing an increase in the passage number of stem cells, compared to known methods of separating cells from a cell culture dish. In addition, since an empty space of the cell culture dish is filled with cells over time, it is possible to continuously supply cells for the polymer-based fiber support without an additional subculture process.

Further, since cells may migrate to the polymer-based nano/microfiber support without external stimulation, artificial influence on cells may be minimized, and thus a differentiation potency of stem cells is enhanced, thereby inducing more effective differentiation. Accordingly, the present invention, as a cell therapy, may be applied to general fields of regenerative medicine and tissue engineering.

### DESCRIPTION OF THE DRAWINGS

FIG. 1 shows a basic concept of the present invention, in which a method of repeatedly inoculating and differentiating cells on a polymer-based nano/microfiber by a cell stamping method is illustrated;

FIG. 2 shows graphs illustrating experimental results of osteogenic differentiation according to the present invention, in which cells were repeatedly inoculated on many different polymer-based nano/microfiber supports by a cell stamping method, and then allowed to differentiate into osteoblasts;

FIG. 3A illustrates a control group which was seeded on a polymer-based nano/microfiber support by trypsin treatment, and FIG. 3B illustrates a group of cells which were inoculated by using the cell stamping method of the present invention;

FIG. 4 shows graphs illustrating a difference in cartilage differentiation between cells (G2) inoculated on the polymer-based nano/microfiber of the present invention by the cell stamping method, and cells (G1) seeded by general trypsin treatment; and

FIG. 5 shows images of fluorescence microscopy of cells which migrated to each of polymer-based nano/microfibers, after each polymer-based nano/microfiber having a high concentration or a low concentration of growth factors conjugated to a surface thereof was placed on a cell culture plate.

### BEST MODE

An aspect relates to a trypsin-free stamp system. In the system, the stamp may include a polymer-based nano/microfiber support.

In the cell stamp system of the present invention, the support may be characterized by being porous for mechanical stability and cell culture. Further, the support may be used for supporting, culturing, or transplanting cells.

The polymer may be one or more selected from the group consisting of gelatin, poly-alpha-ester group (poly-esters group), polyglycolic acid (PGA), polylactide (PLA), poly L-lactic acid (PLLA), poly D-lactic acid (PDLA), poly lactic-co-glycolic acid (PLGA), polycaprolactone (PCL), poly 2-hydroxyethyl methacrylate (pHEMA), polyethylene glycol (PEG), polypropylene glycol (PPG), polyhydroxybutyrate (PHB) which is polyhydroxyalkanoate, polydioxanone (PDO, PDS), polyurethane (PU), polypropylenefumarate (PPF), polyanhydrides, polyacetals, polyorthoesters, polycarbonates, polyphosphazenes, polyphosphoesters, poly N-isopropylacrylamide (PNIPAM), polyacrylamide (PAAm), polyitaconic acid (PIA), dextran, chitosan, alginate, hyaluronic acid, chondroitin sulfate (CS), heparin, keratin, dermatan, gelatin, collagen, albumin, fibrin, cellulose, elastin, poly gamma-glutamic acid, poly L-lysine, poly L-glutamic acid, polyaspartic acid, polysaccharides (starch), lignin, agar, xanthan gum, acacia, carrageenan, sterculia gum, and ispaghula.

The support may be composed of a solid having greater hardness than cells, for example, a gel, but is not limited thereto. Further, the support may include a variety of active factors such as tissue factors, growth factors, drugs, etc., which may help growth and differentiation of cells to be cultured.

In the trypsin-free cell stamp system of the present invention, the cells may be preferably anchorage-dependent cells. The anchorage-dependent cells may be preferably stem cells, and more preferably, mesenchymal stem cells, embryonic stem cells, or induced pluripotent stem cells (iPSCs). The mesenchymal stem cells (MSCs) may refer to multipotent stem cells having an ability to differentiate into various mesodermal cells including bone, cartilage, fat, and muscle cells, or ectodermal cells such as nerve cells. The mesenchymal stem cells may be derived from, but are not limited to, one or more selected from the group consisting of an umbilical cord, umbilical cord blood, bone marrow, fat, muscle, nerves, skin, an amniotic membrane, a chorionic membrane, a decidual membrane, and a placenta.

The cells may be derived from a human, a fetus, or a mammal excluding humans. The mammal excluding humans is more preferably a canine animal, a feline animal, a simian animal, a cow, a sheep, a pig, a horse, a rat, a mouse, a guinea pig, etc., and the origin thereof is not limited.

Another aspect provides a method of supporting cells by using the trypsin-free cell stamp system, the method including the steps of: (a) seeding cells in a cell culture plate, and then culturing the cells; and (b) supporting the cultured cells by contacting the cultured cells with a stamp.

In the method of supporting cells of the present invention, the trypsin-free cell stamp system is the same as described above.

The stamp may preferably include a polymer-based nano/microfiber support.

The polymer may be one or more selected from the group consisting of gelatin, poly-alpha-ester group (poly-esters group), polyglycolic acid (PGA), polylactide (PLA), poly L-lactic acid (PLLA), poly D-lactic acid (PDLA), poly lactic-co-glycolic acid (PLGA), polycaprolactone (PCL), poly 2-hydroxyethyl methacrylate (pHEMA), polyethylene glycol (PEG), polypropylene glycol (PPG), polyhydroxybutyrate (PHB) which is polyhydroxyalkanoate, polydioxanone (PDO, PDS), polyurethane (PU), polypropylenefumarate (PPF), polyanhydrides, polyacetals, polyorthoesters, polycarbonates, polyphosphazenes, polyphosphoesters, poly N-isopropylacrylamide (PNIPAM), polyacrylamide (PAAm), polyitaconic acid (PIA), dextran, chitosan, alginate, hyaluronic acid, chondroitin sulfate (CS), heparin, keratin, dermatan, gelatin, collagen, albumin, fibrin, cellulose, elastin, poly gamma-glutamic acid, poly L-lysine, poly L-glutamic acid, polyaspartic acid, polysaccharides (starch), lignin, agar, xanthan gum, acacia, carrageenan, sterculia gum, and ispaghula.

In the step of (b) supporting the cultured cells by contacting the cultured cells with the stamp in the method of supporting cells, the contacting of the cultured cells with the stamp may be performed by directly placing the cell stamp including the polymer-based nano/microfiber support of the present invention on the cells cultured in the cell culture plate. Further, the supporting of the cells may be performed by culturing the cells for a predetermined period of time while placing the cell stamp on the cells. The culturing for a predetermined period of time may be performed for, for example, 1 day to 7 days, 2 days to 6 days, 2 days to 5 days, or 3 days to 5 days. The culturing for supporting the cells may be performed under the same temperature and medium conditions as in the culturing after seeding the cells in the culture plate. When the cell stamp is placed on the cells, and the cells are cultured for a predetermined period of time, cells attached on the culture plate may migrate to the stamp, and as a result, cells under the stamp may be supported on the stamp.

Still another aspect provides a method of culturing cells by using the trypsin-free cell stamp system, the method including the steps of: (a) seeding cells in a cell culture plate, and then culturing the cells; (b) supporting part of the cultured cells on a stamp by contacting the cultured cells with the stamp; and (c) culturing non-supported cells which remain on the cell culture plate.

In the method of culturing cells of the present invention, the trypsin-free cell stamp system is the same as described above.

In the method of culturing cells of present invention, the step of (b) supporting is the same as described in the supporting method.

In a specific embodiment of the present invention, by using a stamp smaller than a culture plate, only a portion of cultured cells is supported on the stamp, and non-supported remaining cells may exist in the culture plate. When the remaining cells in the culture plate are further cultured, the remaining cells migrate to and grow in an empty space which is generated due to the cells supported on the stamp, such that the empty space is filled with the cells. Accordingly, the method of the present invention may be used to subculture cells without trypsin treatment, unlike known methods of subculturing cells by trypsin treatment.

In the method of culturing cells of the present invention, the steps (b) and (c) may be repeated once or more. As a result, the method of culturing cells of the present invention may be used to continuously grow and culture cells even without trypsin treatment.

In the method of culturing cells of the present invention, a step of further culturing the cells supported on the stamp may be performed. The step of further culturing the cells supported on the stamp may be performed to further culture the supported cells on the stamp by providing the stamp with a culture medium.

Still another aspect provides a method of transplanting cells by using the trypsin-free cell stamp system, the method including the steps of: (a) seeding cells in a cell culture plate, and then culturing the cells; (b) supporting the cultured cells on a stamp by contacting the cultured cells with the stamp; (c) separating the support, on which the cells are supported, from the stamp; and (d) transplanting the separated support into a living body.

In the method of transplanting cells of the present invention, the trypsin-free cell stamp system is the same as described above.

The method of transplanting cells of the present invention may further include a step of culturing or differentiating the cells on the separated support after the step (c).

Further, the support may be transplanted into a living body for cell therapy or tissue regeneration.

In the present invention, a method of repeatedly inoculating and differentiating cells on a polymer-based nano/microfiber by a cell stamping method was confirmed.

In an Example of the present invention, adipose stem cells (ASCs) were inoculated in a cell culture plate and then cultured for 2 to 3 days. A polymer-based nano/microfiber support was placed on the culture plate, in which the cells were cultured, and then fixed with a Teflon holder. 3 to 5 days later, the support was separated and transferred to a new cell culture plate, and then cells were cultured by using a differentiation medium. An empty space generated in the existing culture plate in the form of the fiber support was confirmed to be filled with cells by proliferation for 7 days.

Accordingly, an aspect of the present invention may provide a method of culturing cells by using the support-based trypsin-free cell stamp system, the method including the steps of: (a) seeding cells in a cell culture plate, and then culturing the cells; (b) contacting the cultured cells with the support-based stamp, and then migrating and adhering the cells to the support-based stamp by further culturing the cells; (c) separating the support-based stamp, to which the cells migrate and adhere, and then further culturing the cells; and (d) repeating the steps (b) and (c).

In the present invention, the cells to be cultured refer to cells commonly used in tissue engineering and regenerative medicine. Regenerative medicine is a field that replaces or regenerates human cells, tissues, or organs to restore their original functions, and involves attempts to culture tissues or organs having no self-healing capability in a laboratory and safely transplant the tissues or organs. Since cells to be cultured are transplanted into a living body in the form of a support on which the cells are cultured, the cells are required to have high adhesiveness and affinity for the support.

In the present invention, the cells may be characterized by anchorage-dependent cells, and the anchorage-dependent cells may be characterized by stem cells.

In the present invention, the cells to be cultured may be preferably stem cells. Stem cells may refer to cells that are able to develop into any type of tissue, and are also called mother cells. Stem cells are generally collected from an embryo at an early stage of division. Cells at this stage do not have yet an ability to form an organ, and therefore, may be cultured into a particular cell line which is selected according to a predetermined input. The stem cells may include embryonic stem cells derived from a human embryo; adult stem cells such as bone marrow cells that constantly produce blood cells; mesenchymal stem cells which are multipotent stem cells that have an ability to differentiate into various mesodermal cells including bone, cartilage, fat, and muscle cells, or ectodermal cells such as nerve cells; or induced pluripotent stem cells (iPS cells) derived from human somatic cells. The stem cells may be any stem cells without particular limitation, as long as they may be generally used in tissue engineering and regenerative medicine, and may be cultured into bioorgans and biotissues. The mesenchymal stem cells may be derived from, but are not limited to, an umbilical cord, umbilical cord blood, bone marrow, fat, muscle, nerves, skin, an amniotic membrane, a chorionic membrane, a decidual membrane, or a placenta.

In the present invention, the support is a generic term for extracellular materials that are required for an attachment site and a path where cells divide or form tissues. At present, almost all products of tissue engineering are studied for their affinity with cells, and therefore, in a broad sense, may be expressed as a support that is adjacent to cells and supports the cells. The term support is key to tissue engineering, and studies thereon are extensive. Generally, supports are composed of a solid having greater hardness than cells, and recently, there are many attempts to use various gels. Supports are essential for cell growth, and a growth and differentiation environment of cells to be cultured is governed not only by tissue factors or hormones but also by the shape and components of a support, and may include various active factors such as growth factors, drugs, etc., and may exist in combination with elements other than the support. Therefore, the support is a basic substance of tissue regeneration, which includes all influencing factors, not the isolated cell factors, and has the greatest effect on cells. Eventually, all supports require a porous structure for nutrient exchange by diffusion, unless the support is of a specialized form designed as thin as a gel. In other words, using a support without a porous structure is almost impossible. In this sense, porosity is mentioned as a characteristic of a support, and the support is classified by a size, a shape, interconnectivity, and an orientation of pores.

Moreover, fiber supports have a repeated pattern of a specific form and chemical characteristics, and required conditions vary depending on cells. These materials are biocompatible materials, mimic the three-dimensional extracellular matrix environment of the human body, and provide short-term mechanical stability, a sufficient area for cell migration, etc. due to appropriate porosity. Further, it is ideal that supports have a property whereby cells are slowly absorbed to the supports as the cells grow. Supports are developed to include supplementary substances such as growth factors, cell proliferation materials, drug delivery systems, and traceable factors, and development of supports shows the potential to absorb all areas other than tissue engineering. Fiber supports based on biocompatible and biodegradable natural polymers and synthetic polymers to make up for mechanical strength may be prepared in a patch form through electrospinning, and each polymer material may be exposed to a crosslinking agent for different times to control the degree of crosslinking. A variety of polymer-based fiber supports thus prepared enable more selective and efficient delivery of loaded cells, growth factors, or drugs, depending on a biodegradation rate.

Therefore, in the present invention, the support may be characterized by a porous polymer-based nano/microfiber having greater hardness than cells to be cultured, but is not limited thereto.

In addition to the above gelatin porous polymer-based nano/microfiber, the support of the present invention may be any support without limitation, as long as it is a biocompatible polymer support. For example, a poly-alpha-ester group, polyglycolic acid (PGA), polylactide (PLA) and a kind thereof including poly L-lactic acid (PLLA), poly D-lactic acid (PDLA), poly D,L-lactic acid (PDLLA), poly lactic-co-glycolic acid (PLGA) which is a synthetic material of PGA and PLA, polycaprolactone (PCL), poly 2-hydroxyethyl methacrylate (pHEMA), polyethylene glycol (PEG), polypropylene glycol (PPG), polyhydroxybutyrate (PHB) which is polyhydroxyalkanoate, polydioxanone (PDO, PDS), polyurethane (PU), polypropylenefumarate (PPF), polyanhydrides, polyacetals, polyorthoesters, polycarbonates, polyphosphazenes, polyphosphoesters, poly N-isopropylacrylamide (PNIPAM), polyacrylamide (PAAm), polyitaconic acid (PIA), a natural polymer including dextran, chitosan, alginate, hyaluronic acid, chondroitin sulfate (CS), heparin, keratin, dermatan, gelatin, collagen, albumin, fibrin, cellulose, elastin, poly gamma-glutamic acid and poly L-lysine which are kinds of natural polyamino acids, poly L-glutamic acid and polyaspartic acid which are kinds of synthetic polyamino acids, polysaccharides (starch), lignin, agar, xanthan gum, acacia, carrageenan, sterculia gum, ispaghula, etc. may be used.

In the present invention, the cells to be cultured may be attached to the support and then transferred without treatment of trypsin which is used in known methods of culturing cells. Trypsin is a proteolytic enzyme of pancreatic juice. An inactive precursor trypsinogen is produced by the pancreas, and found in the pancreatic juice. When trypsinogen is transferred to the small intestine, it is activated by either enterokinase or trypsin itself, and converted to trypsin. Trypsin is the most important enzyme in digesting proteins, along with pepsin. Due to these characteristics, trypsin is used in a cell culture process and functions to detach cells. However, in the present invention, the support is placed in a culture plate on which cells are cultured, and 3 to 5 days later, the support, to which the cells adhere, is separated. This procedure takes the role of trypsin. Thereafter, the separated support is cultured and differentiated in a new cell culture plate to undergo a preparation process for cell therapy and tissue regeneration.

In the present invention, the cells of the support-based stamp which is separated in the step (c) may be characterized by being further cultured to be used in cell therapy and tissue regeneration.

In the present invention, the cell stamping support is mainly composed of collagen which is the most abundant protein in the tissues of mammals, and may be used as a source material for all organ and tissue regeneration in the form of gauze during surgical operation. Further, such support may be loaded with a drug to induce differentiation of stamped stem cells into desired cells, and therefore, may maximize a regeneration effect of using stem cells through sustained release. In the treatment of many diseases such as lumbar disc herniation, spinal stenosis, scoliosis, vertebral fracture, vertebral tumor spinal deformity, spinal trauma, etc., which are musculoskeletal diseases of the spine, it is possible to apply spinal fusion, which removes the inter-segmental motion and maintains steady stability, by incorporation of stamped stem cells and a host bone, instead of existing spinal fixation devices.

Further, the support may be used in regeneration of cartilage for the treatment of a disease associated with articular cartilage damage, such as rheumatoid arthritis, traumatic articular cartilage injury, cartilage fracture, chondromalacia, etc. It is possible to maximize the effect of cartilage regeneration by adhering a damaged cartilage area while maintaining an extracellular matrix through a single operation, instead of osteochondral transplantation and autologous chondrocyte transplantation which are known cartilage repair methods that require repeated operations, or bone marrow stimulation (microfracture and abrasion) which generally stimulates fibrocartilage repair.

The stamping technique may be applied to esophageal reconstruction. Recently, endoscopic submucosal dissection (ESD), which is a surgical procedure for removing esophageal cancer, has been used. However, after removal of esophageal cancer, inflammation and stenosis develop on the surface of the esophagus, and thus a secondary treatment is needed. Accordingly, a highly adhesive trypsin-free stamping patch is transplanted into the damaged site after removal of the esophageal cancer, while preserving the mucosal cells and their extracellular matrix (ECM) without damage, and thus may be used as an alternative therapy for relieving an inflammatory reaction or stenosis.

The stamping technique may also be applied to cardiovascular regeneration. Myocardial infarction, which is one of the cardiovascular diseases, is a disease in which the myocardial wall becomes thin or does not function due to fibrosis, and cardiac transplantation or cell transplantation, medication, etc. is known as a major therapy. In 2007, W. R. Wagner and colleagues reported that a patch-type support was transplanted into the heart of myocardial infarcted rats to relieve myocardial infarction by cardiovascular regeneration (Kazuro L. Fujimoto, J Am Coll Cardiol, 49(23), 2292-2300, 2007). As compared to the support of the research team, the stamping patch may be adjusted in thickness, which has little effect on the mobility of the heart, and may be adhered without suturing for support fixation, thereby enhancing the effect of cardiovascular regeneration.

In addition, the stamping technique may also be applied to nerve regeneration. Common spinal cord injuries are caused by spinal subluxation due to trauma, or spinal cord compression due to tightening of the spinal canal such as through vascular injury, spinal myelopathy, tumors, etc., and many different symptoms occur depending on the degree of damage. Since nerve cells grow only up to 1˜3 mm per day, methods for supplying cells and drugs to the site of injury have been suggested for more efficient regeneration. The stamping patch of the present invention may be applied by a simplified process and may have excellent bioactivity, and therefore, may be used for the treatment of incomplete nerve injury and the restoration of the functions through suppression of nerve cell necrosis and reactive oxygen production, and facilitation of nerve differentiation, leading to more rapid therapeutic results.

### Mode of the Invention

Hereinafter, the present invention will be described in more detail with reference to Examples. However, these Examples are for illustrative purposes only, and it will be apparent to those skilled in the art that the scope of the present invention is not intended to be limited by these Examples.

### Example 1

Preparation of Polymer-Based Nano/Microfiber Support

To prepare a polymer-based nano/microfiber support, an electrospinning system was used. First, a polymer to be used in the fiber support may be prepared as a solution. If a crosslinking process is necessary, the crosslinking process may be performed. In the case of a gelatin-based nano/microfiber support, 12 wt % of gelatin was dissolved in formic acid and prepared as a solution. A first crosslinking process was performed by adding 1 wt % of glutaraldehyde (GTA) to the gelatin solution. The polymer prepared as the solution was transferred to a needle and a syringe for electrospinning, and the polymer solution was subjected to electrospinning according to appropriate conditions. To prepare the gelatin-based nano/microfiber support, 0.7 mL of the solution per time was spun, a voltage was 16.0 kV, a spinning speed was 2000 mm/min, and a distance between a collector and the needle was 10 cm. The conditions of the electrospinning system vary depending on properties of a polymer, and a thickness of fiber strands constituting a polymer-based fiber support may be controlled in a nano/micrometer range depending on the state of a polymer solution. Subsequently, when the fibers are considered to be unsuitable for the support, they are subjected to a secondary crosslinking process. To prevent short-term biodegradation of the gelatin-based nano/microfiber support, crosslinking was performed for 1 hour by using glutaraldehyde (GTA) which is the same crosslinking agent as used above. Then, to eliminate toxicity of glutaraldehyde (GTA), the fibers were washed with glycine for 12 hours.

### Example 2

2-1. Method of Culturing Cells by Using Trypsin-Free Cell Stamp System

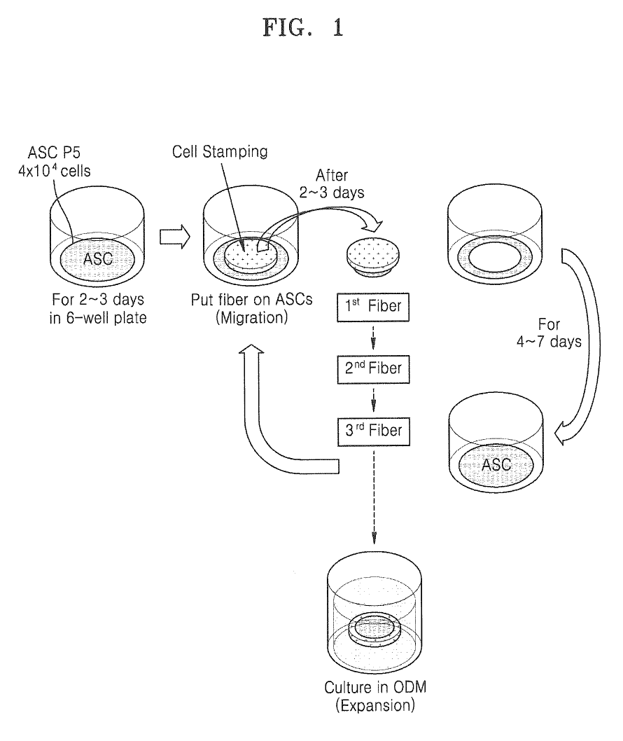

Adipose stem cells (ASCs) were seeded in a 6-well cell culture plate, and then cultured for 2 days˜3 days. Next, the polymer-based nano/microfiber support was placed in the plate well to which the cells were adhered, and the gelatin fibers were fixed with a teflon holder. 3 days˜5 days later, the polymer-based nano/microfiber support was separated and transferred to a new cell culture plate, followed by incubation with a differentiation medium. An empty space in the 6-well plate, which was formed in the shape of the fiber support after migration of the cells, was found to be filled with the cells by proliferation after about 7 days (FIG. 1).

2-2. Isolation and Culturing of Human Adipose-Derived Stem Cells

Under the permission of the Ethics Committee of CHA hospital, CHA University School of Medicine, informed consent was obtained from patients, and adipose tissues removed by liposuction were collected. To remove contaminant blood, the collected adipose tissues were washed with a phosphate buffered saline (PBS, Sigma, St. Louis, Mo.) three times. Subsequently, the adipose tissues were digested with 0.2 w/v % bovine serum albumin-supplemented PBS and 2 mg/mL type-II collagenase (Sigma) for 45 minutes at 37° C. The digested tissues were filtered with a 70 μm-filter, and a filtrate was centrifuged to remove suspended adipose cells. The isolated adipose-derived stem cells (ASCs) were incubated in a stem cell culture medium [1% penicillin-streptomycin and 10% fetal bovine serum (FBS)(Invitrogen, USA)-supplemented Dulbecco's modified Eagle medium (DMEM, Gibco BRL, Gaithersburg, Md.)] at 37° C. and 5% CO₂.

### Example 3

3-1. Experiment of Cell Differentiation by Using Trypsin-Free Cell Stamp System

To inoculate cells by a cell stamping method, different numbers (G1˜G5) of adipose stem cells (ASCs) were seeded in 6-well cell culture plates and cultured for 2 days˜3 days. Next, the nano/microfiber support prepared in Example 1 was placed in the plate well to which cells adhered, and cultured for 2 days˜3 days. This procedure was repeated three times. In this regard, a first nano/microfiber support was separated from the cell culture plate, and cells were allowed to proliferate for 4 days˜7 days. Then, a second nano/microfiber support was placed into the cell culture plate, followed by stamping inoculation. The nano/microfiber supports on which cells were inoculated by a trypsin-free cell stamping method were incubated in an osteogenic differentiation medium for 7 days. After completion of the incubation, RNA was isolated with a trizol reagent, and then cDNA was synthesized by RT-PCR. cDNA obtained from each fiber support was used to quantify osteogenic differentiation marker genes in the cells obtained from the cell stamp system by q-PCR, in order to analyze a degree of differentiation.

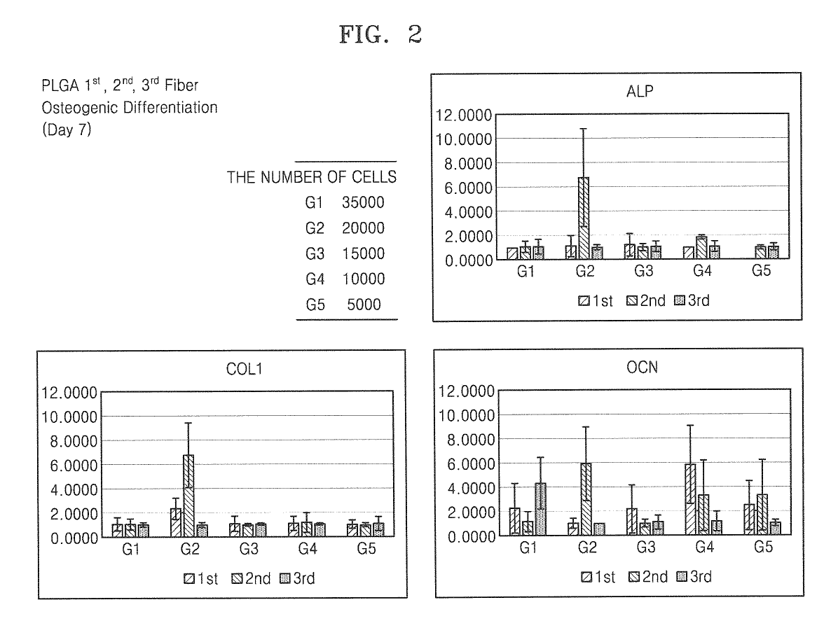

As a result, in the cells inoculated by the cell stamp system, osteogenic differentiation marker genes (ALP, COL1, OCN) were observed, indicating osteogenic differentiation (FIG. 2). Accordingly, it was confirmed that normal cell differentiation occurs when using the cell stamp system.

3-2. Comparison of Cartilage Differentiation Degree by Trypsin-Free Cell Stamp System

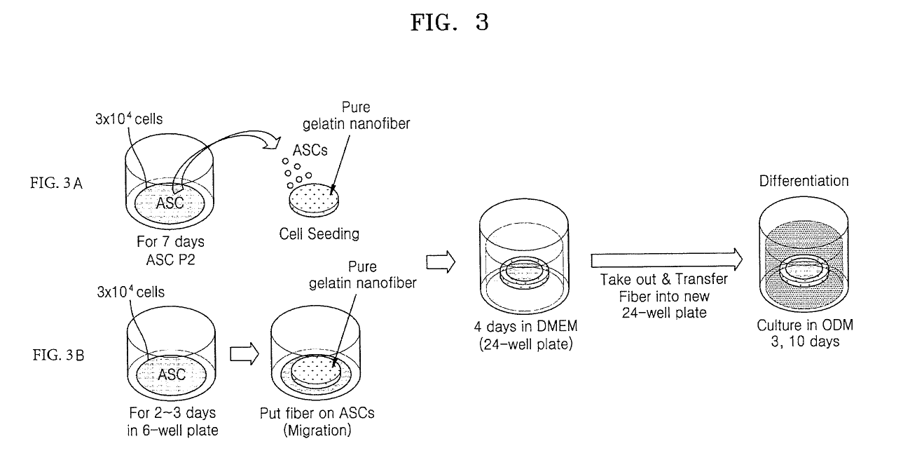

Adipose stem cells (ASCs) were seeded in a cell culture plate (6-well plate), and then cultured for 2 days˜3 days. Next, as a control group, cells were treated with trypsin and seeded on a polymer-based nano/microfiber support (FIG. 3, A). As another group, cells were inoculated by a cell stamping method of putting gelatin fibers in a plate well to which cells adhered, and then fixing the gelatin fibers with a teflon holder (FIG. 3, B). The polymer-based nano/microfibers on which cells were inoculated by the above-mentioned two methods were expanded for 4 days in a general medium, and then transferred to new cell culture plates (24-well plate) and cultured with a cartilage differentiation medium for 3 days and 10 days. After the culturing for cartilage differentiation was completed, RNAs were isolated by using a Trizol reagent, and cDNAs were synthesized by RT-PCR. cDNA obtained from each fiber support was used to quantify osteogenic differentiation marker genes by q-PCR, and thereby the degree of differentiation was compared between the method of inoculating cells by using the cell stamping method and the method of inoculating cells by seeding, wherein the seeding method is a general method of inoculating cells.

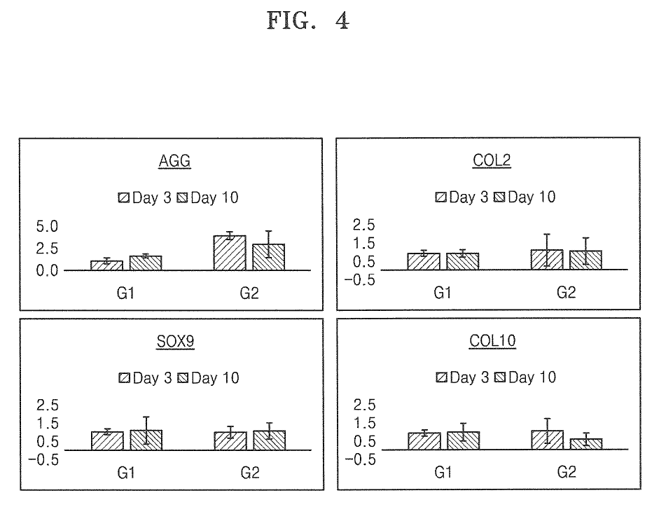

As a result, higher expression of early marker genes (AGG, COLII) of cartilage differentiation was observed in the cells inoculated by the cell stamp system (FIG. 4, G2) than in the cells seeded after trypsin treatment (FIG. 4, G1), indicating improved cartilage differentiation (FIG. 4). Accordingly, it was confirmed that use of the cell stamp system improves cell differentiation potency, compared to trypsin treatment.

### Example 4

Test of Cell Migration According to Surface Properties of Supports

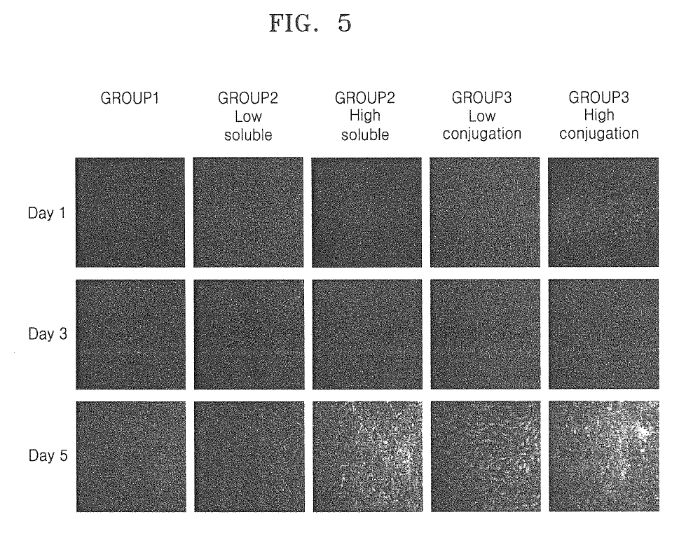

To vary properties of the surface of the support, growth factors were conjugated to the surface of polymer-based nano/microfibers. Experiments were performed by dividing into non-treated polymer-based nano/microfibers (Group 1), a soluble growth factor-added group (Group 2), a growth factor-conjugated polymer support (Group 3). Concentrations of the growth factor used in Groups 2 and 3 were two concentrations of a high concentration and a low concentration. Polymer-based nano/microfibers (Group 1) were placed in the cell culture plate, to which cells were adhered. After placing the polymer-based nano/microfibers, soluble growth factors were injected (Group 2). A high concentration or a low concentration of growth factors was conjugated to the surfaces of the polymer-based nano/microfibers, and then each of the conjugated polymer-based nano/microfibers was placed in the cell culture plate, to which cells were adhered, and cultured for 1 day, 3 days, or 5 days. Cells which migrated to each of the fibers were stained and observed under a fluorescence microscope (FIG. 5).

In FIG. 5, when Group 1, Group 2 with high solubility, and Group 3 with high conjugation are compared with each other, migration of a large amount of cells is observed in the polymer-based nano/microfibers, of which a surface was conjugated with growth factors, as compared to the non-treated polymer-based nano/microfibers. Further, when Group 3 with low conjugation and Group 3 with high conjugation are compared with each other, migration differs depending on the concentration of the growth factors conjugated to the surface of the support. Accordingly, it was confirmed that migration may differ depending on the surface of the support.

As described above, the specific portion of the present invention was described in detail. It will be obvious to a person having ordinary skill in the art that specific description is only a description of the preferred embodiment and is not to be construed to limit the scope of the present invention. Thus, the substantial scope of the present invention will be defined by the appended claims and equivalents thereof.

1. A trypsin-free cell stamp system.

2. The system of claim 1, wherein the stamp comprises a polymer-based nano/microfiber support.

3. The system of claim 2, wherein the support is porous for mechanical stability and cell culture.

4. The system of claim 2, wherein the support is used for supporting, culturing, or transplanting cells.

5. The system of claim 2, wherein the polymer is at least one selected from the group consisting of gelatin, poly-alpha-ester group (poly-esters group), polyglycolic acid (PGA), polylactide (PLA), poly L-lactic acid (PLLA), poly D-lactic acid (PDLA), poly lactic-co-glycolic acid (PLGA), polycaprolactone (PCL), poly 2-hydroxyethyl methacrylate (pHEMA), polyethylene glycol (PEG), polypropylene glycol (PPG), polyhydroxybutyrate (PHB) which is polyhydroxyalkanoate, polydioxanone (PDO, PDS), polyurethane (PU), polypropylenefumarate (PPF), polyanhydrides, polyacetals, polyorthoesters, polycarbonates, polyphosphazenes, polyphosphoesters, poly N-isopropylacrylamide (PNIPAM), polyacrylamide (PAAm), polyitaconic acid (PIA), dextran, chitosan, alginate, hyaluronic acid, chondroitin sulfate (CS), heparin, keratin, dermatan, gelatin, collagen, albumin, fibrin, cellulose, elastin, poly gamma-glutamic acid, poly L-lysine, poly L-glutamic acid, polyaspartic acid, polysaccharides (starch), lignin, agar, xanthan gum, acacia, carrageenan, sterculia gum, and ispaghula.

6. The system of claim 1, wherein cells thereof are anchorage-dependent cells.

7. The system of claim 6, wherein the anchorage-dependent cells are stem cells.

8. The system of claim 6, wherein the cells are mesenchymal stem cells, embryonic stem cells, or induced pluripotent stem cells (iPSCs).

9. A method of supporting cells by using a trypsin-free cell stamp system, the method comprising the steps of:
(a) seeding cells in a cell culture plate, and then culturing the cells; and
(b) supporting the cultured cells by contacting the cultured cells with a stamp.

10. The method of claim 9, wherein the stamp comprises a polymer-based nano/microfiber support.

11. The method of claim 9, wherein the polymer is at least one selected from the group consisting of gelatin, poly-alpha-ester group (poly-esters group), polyglycolic acid (PGA), polylactide (PLA), poly L-lactic acid (PLLA), poly D-lactic acid (PDLA), poly lactic-co-glycolic acid (PLGA), polycaprolactone (PCL), poly 2-hydroxyethyl methacrylate (pHEMA), polyethylene glycol (PEG), polypropylene glycol (PPG), polyhydroxybutyrate (PHB) which is polyhydroxyalkanoate, polydioxanone (PDO, PDS), polyurethane (PU), polypropylenefumarate (PPF), polyanhydrides, polyacetals, polyorthoesters, polycarbonates, polyphosphazenes, polyphosphoesters, poly N-isopropylacrylamide (PNIPAM), polyacrylamide (PAAm), polyitaconic acid (PIA), dextran, chitosan, alginate, hyaluronic acid, chondroitin sulfate (CS), heparin, keratin, dermatan, gelatin, collagen, albumin, fibrin, cellulose, elastin, poly gamma-glutamic acid, poly L-lysine, poly L-glutamic acid, polyaspartic acid, polysaccharides (starch), lignin, agar, xanthan gum, acacia, carrageenan, sterculia gum, and ispaghula.

12. A method of culturing cells by using a trypsin-free cell stamp system, the method comprising the steps of:
(a) seeding cells in a cell culture plate, and then culturing the cells;
(b) supporting a portion of the cultured cells on a stamp by contacting the cultured cells with the stamp; and
(c) culturing non-supported cells which remain on the cell culture plate.

13. A method of transplanting cells by using a trypsin-free cell stamp system, the method comprising the steps of:
(a) seeding cells in a cell culture plate, and then culturing the cells;
(b) supporting the cultured cells on a stamp by contacting the cultured cells with the stamp;
(c) separating the support, on which the cells are supported, from the stamp; and
(d) transplanting the separated support into a living body.

14. The method of claim 13, further comprising a step of culturing or differentiating the cells on the separated support after the step (c).

15. The method of claim 13, wherein the support is transplanted into a living body for cell therapy or tissue regeneration.
